(19) United States
(12) Patent Application Publication
(10) Pub. No.: US 2018/0317912 A1
(43) Pub. Date: Nov. 8, 2018

(54) Dual Action Surgical Thread

(71) Applicant: Marco Brandi, Miami, FL (US)

(72) Inventor: Marco Brandi, Miami, FL (US)

(21) Appl. No.: 15/973,151

(22) Filed: May 7, 2018

Related U.S. Application Data
(60) Provisional application No. 62/502,895, filed on May 8, 2017.

(51) Int. Cl. A61B 17/06

(52) U.S. Cl. CPC A61B 17/06166; A61B 2017/00004; A61B 2017/06185; A61B 2017/06176

(57) ABSTRACT

A dual action surgical thread is provided. The thread is designed for use in general surgical and cosmetic surgery applications where a lifting or tensioning of skin is desired. The thread allows for lifting action along an entire inserted length by providing parallel sections of lifting and anchoring along a folding zone, where a distal end of the thread is folded back along the shaft of the thread.

### FIELD OF THE INVENTION

The present invention relates to a dual action surgical thread and methods of soft tissue strengthening or repair. The thread is used to provide both lifting and anchoring functions when inserted into a dermal or hypodermal layer of skin. The thread is useful in general surgical procedures and also has applicable usage in the field of cosmetic surgery, on both an inpatient and an outpatient basis.

### BACKGROUND OF THE INVENTION

Recent advances in the surgical arts have resulted in the widespread use of dissolvable threads in surgical procedures. The obvious advantage of dissolvable suture threads is that the patient's body can absorb the suture material during the healing process, thus eliminating the need for follow-up care to cut and remove non-dissolving sutures.

In the field of cosmetic surgery, the prior art has evolved to use dissolvable threads with barbs or cogs that provide a lifting action where a patient's skin can be pulled or shaped in a certain direction with the reverse direction being resisted by the opposing action of the thread barbs. By using dissolvable thread with barbs, the medical and cosmetic practitioner can affect a desired skin-tensioning outcome on a patient by inserting a barbed dissolvable thread and leaving the thread in the dermal or hypodermal layer of the skin for reabsorption into the body after setting proper tension and the desired direction of skin pull. One advantage of using dissolvable barbed threads for cosmetic procedures is that during the healing process the body naturally produces collagen around the inserted thread. This naturally produced collagen forms a connective structure of supported skin that provides a dermal architecture to maintain the desired pull or positioning of the skin following the body's absorption of the barbed thread. In this manner, barbed dissolvable threads are useful in providing an immediate cosmetic lift and in promoting the skin to heal in a desired shape over the long term.

Using dissolvable barbed threads in cosmetic procedures is simple and relatively painless. The threads are inserted below the epidermal layer using small cannulas preloaded with thread of the desired barb configuration. When the delivery cannula is removed, the dermal entrance hole quickly closes over. Thus, dissolvable threads eliminate the need for painful incisions typically used in cosmetic procedures such as facelifts or necklifts, incisions that can become infected, that take time to heal, and that often leave behind small scars that must be hidden in the folds of the patient's skin.

One challenge that existed with dissolvable threads prior to the present invention was how to provide a strong lifting action across the length of the thread body while also maintaining sufficient anchor on the thread so that the desired skin tension can be maintained during the complete healing and thread dissolving process. Existing thread barb configurations use juxtaposed sections of lifting and anchoring so that a proximal portion of the thread provides lift while a distal portion of the thread provides anchoring, or visa versa. For example, U.S. Pat. App. No. 2010/0234947 discloses contiguous sections of dissolvable thread with barbs running in opposing directions, thus presenting two sections, one for lift and one for anchoring. This configuration is well-known in the prior art. The problem with this arrangement is that it may be desirable to provide lifting across the entire length of where the thread is to be inserted. This is not possible where a distal section lifts and a proximal section anchors, or visa versa. Further, the situation cannot be resolve by placing multiple threads within the same zone of skin because the skin becomes contorted or twisted due to parallel opposing actions of the threads.

There exists a need in the art for a dissolvable surgical thread that can both lift and anchor within the same area of skin treatment. By providing a dual action surgical thread, the present invention allows the medical practitioner to more accurately sculpt and shape the treated area of skin while reducing the need for placing multiple threads within the same dermal area. This minimizes the complexity of the procedure and promotes a quicker healing of the affected area and also allow quicker natural production of a supporting collagen architecture.

### SUMMARY OF THE INVENTION

The invention comprises a surgical thread having a columnar shaft with barbs placed in an angled configuration radiating outward from the shaft. The thread has a segment located above a spreading zone and a segment located below the spreading zone. The barbs of these segments are oriented in opposing directions. The thread is folded back along itself at a folding zone located in the second segment to create an area of dual lifting and anchoring.

### BRIEF DESCRIPTION OF THE DRAWINGS

FIG. 1 illustrates a front view of the surgical thread of the present invention showing the thread in a fully extended length.

FIG. 2 illustrates a front view of the surgical thread of the present invention showing the thread in a folded configuration.

FIG. 3 illustrates a front view of the surgical thread of the present invention loaded in a cannula and ready for delivery in a subcutaneous setting.

FIG. 4 illustrates a front view of the surgical thread of the present placed under the skin.

It will be appreciated that the drawings are illustrative and not limiting of the scope of the invention which is defined by the appended claims. The embodiments shown accomplish various aspects and objects of the invention. It is appreciated that it is not possible to clearly show each element and aspect of the invention in a single figure, and as such, multiple figures are presented to separately illustrate the various details of the invention in greater clarity.

### DETAILED DESCRIPTION OF THE INVENTION

Reference will now be made in detail to the presently preferred embodiments of the invention. There are also representative examples of the invention illustrated in the accompanying drawings. Throughout the following detailed description, the same reference numeral refers to the same elements in all figures.

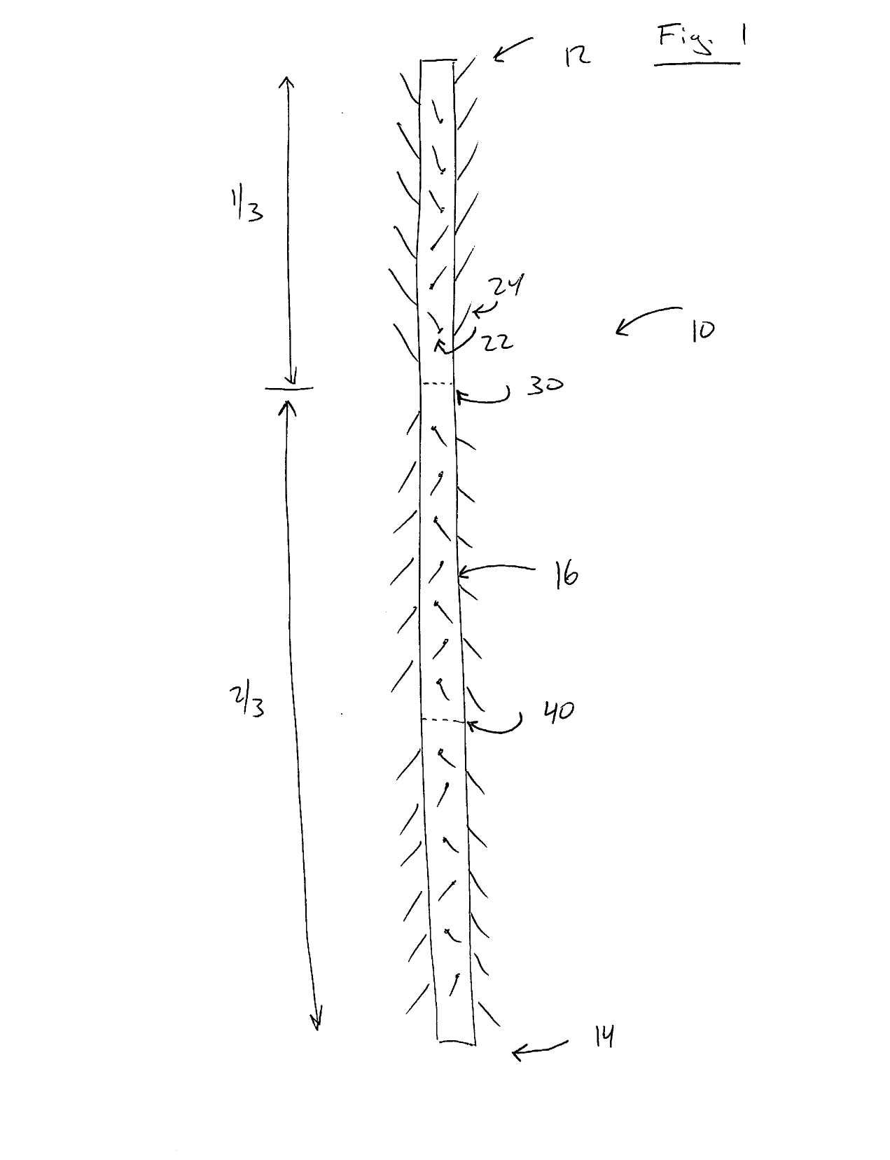

The present invention provides a dual action surgical thread that both lifts and anchors along the same inserted length in a subcutaneous area of the body. Referring to FIG. 1, there is a length of a dissolvable surgical thread 10, which can be made of any soft, resilient and bio-compatible material. In a preferred embodiment thread 10 is made from dissolvable polydiaxonone, which is a biodegradable material that can slowly degrade in vivo over a period of time ranging between about 6 months to about 24 months. Further, thread 10 may be made of varying grades and thicknesses, depending on the amount of lift the practitioner wishes to apply to the affected area of skin. In one embodiment the thread thickness is between a 0.30 mm diameter (3.0 gauge) and a 0.35 mm diameter (2.0 gauge), which is a typical range for suture sizes. Thread 10 has a proximal end 12 and a distal end 14. Proximal end 12 is located furthest from the point of insertion when the thread is first put into the body. Thread 10 also has a columnar shaft 16. The cross section of shaft 16 may be circular or of any other shape that provides support when a force is applied along the length of the shaft.

A plurality of barbs 20 extend in a radial direction outward from shaft 16. The barbs have a base 22 that is connected or formed into shaft 16 and a tip 24 that is distal of the barb base. The barbs are spaced axially from each other and may extend symmetrically or asymmetrically around the cross-sectional shape of shaft 16. In one embodiment of the invention, shaft 16 has a cross-sectional shape of a circle. Other possible cross-section shapes include triangles, squares, pentagons, hexagons, and any other number of sided polygons. Other cross-sectional shapes for shaft 16 include star patterns and other like configurations that provide vertical strength. In the case of a circular cross-section, barbs 20 can include two, three, or more prongs extending symmetrically or asymmetrically about shaft 16. Preferably, the prongs are positioned at 90°, 120°, or 180° from each other. Of course, any number of barb placements can be envisioned that would accomplish a lifting or stopping movement of thread 10. It is also known in the art that barb tip 24 should be positioned at some angle between 1° and 90° from the longitudinal plane of shaft 16 such that the barb tips will hook into or pull against the surrounding medium when thread 10 is pulled in one direction and that such movement of thread 10 is not impeded when the thread is pulled or pushed in an opposite direction.

In accordance with the present invention, thread 10 is divided into three equal-sized lengths marked by two different zones along shaft 16. The first zone, which is located ⅓ of the distance down shaft 16 from distal end 14, is referred to as spreading zone 30. The second zone, which is located ⅔ of the distance down shaft 16 from distal end 14, is referred to as a folding zone 40. Spreading zone 30 marks the transition from barbs oriented in one direction to barbs oriented in an opposite direction. Spreading zone 30 is of sufficient length that the barbs located on either side of the zone do not interfere with each other. Furthermore, the barbs on opposing sides of spreading zone 30 have barb tips 24 that point away from each other. As will be explained below, the spreading zone is beneficial in setting thread 10 in a desired position under the patient's epidermal skin layer. Spreading zone 30 can be of any length that provides for a non-interfering transition between barbs of opposing direction. However, in one embodiment of the invention, spreading zone 30 is from 1.5 cm to approximately 2.5 cm in length, which is of sufficient length to accommodate an ordinary human finger. If the surgical thread of the present invention is formed with a spreading zone sized to accommodate a human finger, then it is beneficial if the zone not have any disposed barbs.

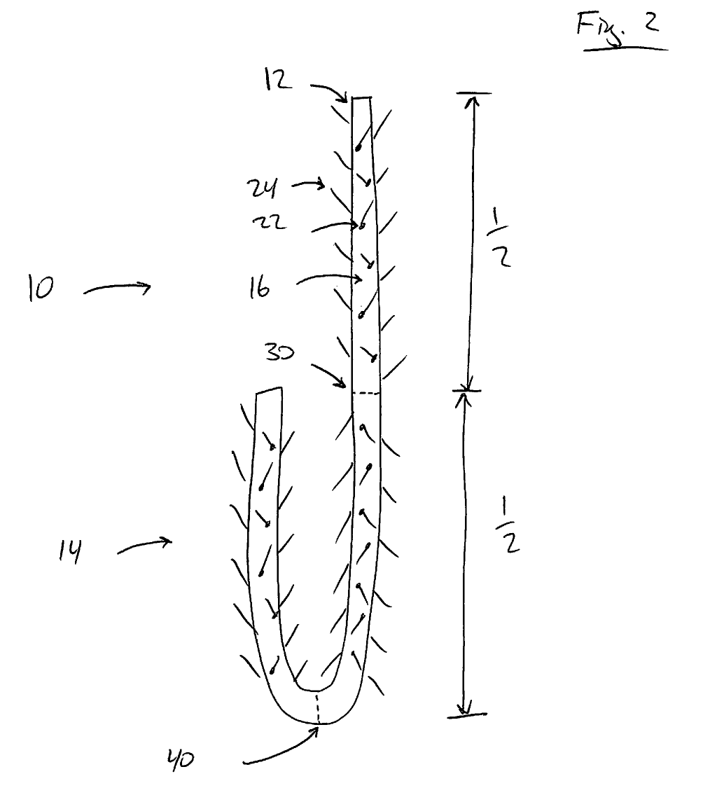

Folding zone 40 is located ⅔ down the length of shaft 16. With this placement of the folding zone, thread 10 can be folded back on itself such that the functional length of the thread as folded comprises two sections of equal length measuring ⅔ of the length of the original, unfolded thread. According to the present invention, all barbs 20 located distal of spreading zone 30 are configured with tips 24 oriented in a direction opposite of the barb tips proximal of the spreading zone. In this manner, thread 10, when folded, will comprise two sections: a proximal section with barbs 20 oriented in a proximal direction, and two essentially parallel distal sections with tips 24 oriented in a distal direction and in a proximal direction (as shown in FIG. 2).

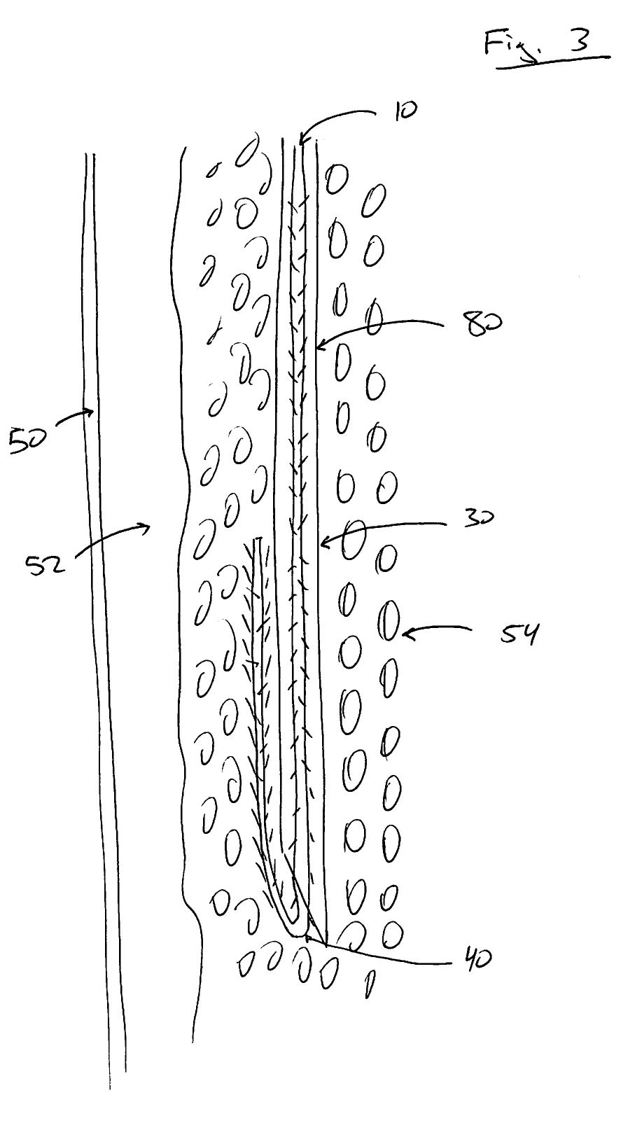

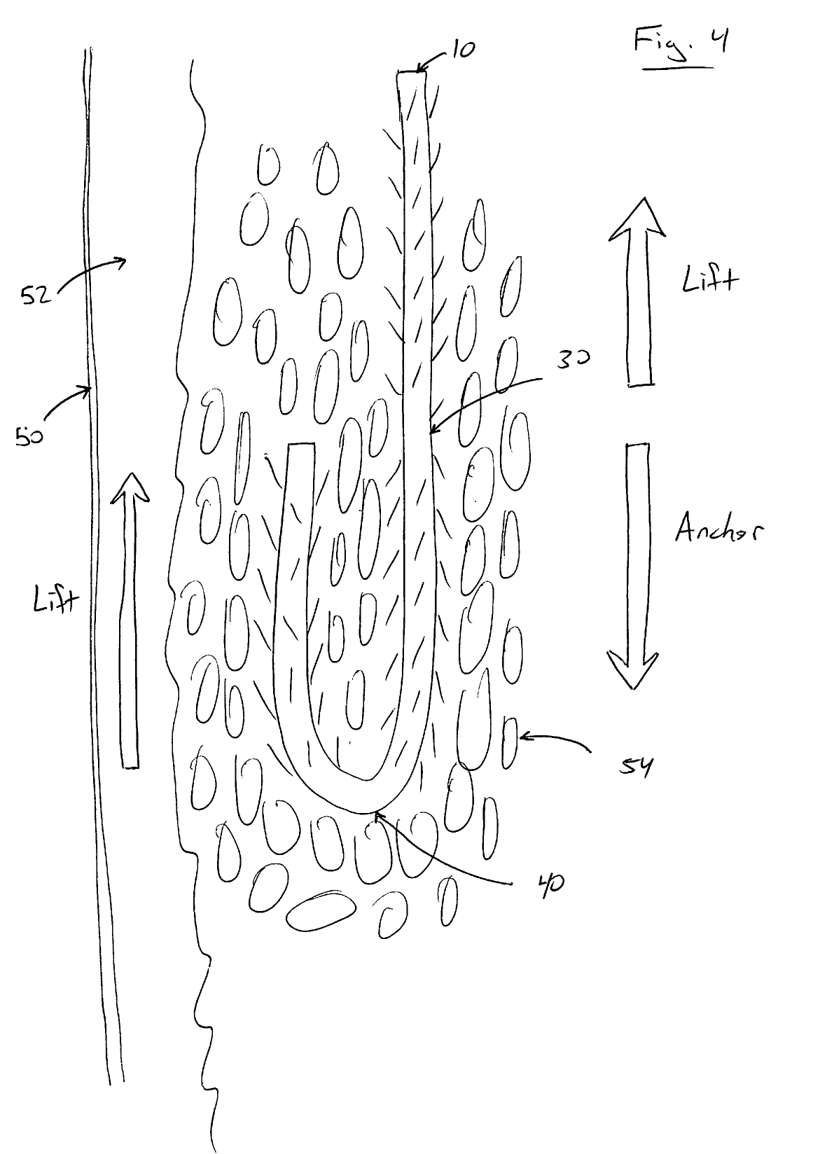

Referring to FIGS. 3 and 4, the placement of the present dual action surgical thread within a patient will now be explained. The medical practitioner takes a section of thread 10 and folds it at folding zone 40 to place distal end 14 backwards in a direction pointing toward spreading zone 30. After making the fold, the practitioner then inserts the folded thread 10 into the lumen of tipped cannula 80. Cannula 80 is tipped with a piercing point for breaking through the patient's epidermis 50. With thread 10 loaded into cannula 80, the practitioner then guides cannula 80 under the patient's epidermis 50 and into the dermis 52 and/or the hypodermis 54 layers of skin. Using the tip of cannula 80 as a guide, the practitioner sets the furthest point distal of the entrance into the skin in which to place folding zone 40 of thread 10. Once the desired location is reached, the practitioner withdraws cannula 80 from the skin, leaving a trail length (not shown) of thread 10 outside the skin for affecting the desired anchoring and lifting action of the thread. The practitioner then massages the outside of the patient's skin to feel for proper placement of the device and to stimulate full engagement between barbs 20 and the underlying skin region. With an understanding that spreading zone 30 is located approximately halfway proximal of the entrance hole, the practitioner can massage the skin both above and below spreading zone 30 to ensure that the anchoring and lifting forces are sufficient to achieve the desired sculpting action. The practitioner also can use the trailing end of thread 10 to pull and tension the thread as desired. When the final sculpting effect is achieved, the practitioner tensions thread 10 to create an elastic effect, cuts the trailing end of thread 10, and then allows thread 10 to slide fully back into the patient's skin at the desired final tension.

By folding distal end 14 back against shaft 16 of thread 10, a dual action region of both lifting and anchoring is created between spreading zone 30 and folding zone 40. The present invention utilizes the region of barbs 20 distal of spreading zone 30 and proximal of folding zone 40 as an anchoring region while the other regions of shaft 16 provide lifting action. Under the barb configuration of the present invention, the fold creates sections of shaft 16 on either side of folding zone 40 having barb tips 24 positioned in opposite directions. Thus, shaft 16 is divided in three contiguous sections of lift, anchor and lift, which allows for better control over the desired skin tensioning and facilitates lifting action across the full inserted length of thread 10.

In one embodiment of the present invention, thread 10 can be preloaded into cannula 80 prior to the procedure to facilitate quicker insertion and deployment of the dual action features of the device.

It will also be appreciated by those of skill in the art that the anchoring and lifting features of the present dual-action device can be customized according to procedural need by adjusting the barb size, the thickness of shaft 16, the cross-sectional configuration of shaft 16, and the general elasticity of thread 10.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes that come within the meaning and range of equivalency of the claims are to be embraced within their scope.

What is claimed is:

1. A dissolvable surgical thread comprising:
a columnar shaft;
barbs placed in an angled configuration radiating outward from said shaft;
a first segment of said shaft located proximal to a spreading zone; and
a second segment of said shaft located distal to said spreading zone;
wherein the barbs of said first and second segments are oriented in opposing directions and a portion of said second segment can be folded along itself at a folding zone to create a dual-action mechanism for lifting and anchoring along said second segment when said shaft is inserted into skin.

2. The dissolvable surgical thread of claim 1, wherein said spreading zone comprises a gap between the barbs of said first and second segments.

3. The dissolvable surgical thread of claim 2, wherein said spreading zone is at least 1.5 cm.

4. The dissolvable surgical thread of claim 1, wherein said second segment is twice the length of said first segment.

5. The dissolvable surgical thread of claim 4, wherein said folding zone is located approximately at the midpoint of said second segment.

6. The dissolvable surgical thread of claim 1, wherein said folding zone comprises a gap between the barbs of said second segment.

7. A method of sculpting skin comprising the steps of:
folding a section of dissolvable surgical thread having angled barbs oriented in substantially the same direction along itself at a folding zone to create an area of dual-action for both lifting and anchoring;
loading said surgical thread at said folding zone into a cannula;
inserting said cannula into a patient's skin;
deploying said thread into said skin; and
massaging said skin to engage said barbs into a dual action of lifting and anchoring in a direction proximal said folding zone.
